(19) United States
(12) Patent Application Publication
(10) Pub. No.: US 2026/0308098 A1
(43) Pub. Date: Oct. 8, 2026

(54) PROCESS FOR IMPROVING RHODIUM/PHOSPHITE-BASED HOMOGENOUS HYDROFORMYLATION CATALYST ACTIVITY

(71) Applicant: Dow Technology Investments LLC, Midland, MI (US)

(72) Inventor: Christophe R. Laroche, Barlesville, OK (US)

(21) Appl. No.: 19/482,449

(22) PCT Filed: Jun. 3, 2024
(86) PCT No.: PCT/US2024/032206

§ 371 Date: Nov. 7, 2025

Related U.S. Application Data
(60) Provisional application No. 63/517,458, filed on Aug. 3, 2023.

(51) Int. Cl. C07C 45/50; B01J 31/18; B01J 31/32

(52) U.S. Cl. CPC C07C 45/505; B01J 31/185; B01J 31/32; B01J 2231/321; B01J 2531/822

(57) ABSTRACT

The present invention relates to hydroformylation processes for producing aldehydes comprising: contacting in a reaction zone, reactants comprising an olefin, hydrogen and CO in the presence of a rhodium-organophosphite based catalyst, optionally with free organophosphite ligand, in the presence of a soluble Group VII metal complex additive, wherein the Group VII complex additive is present at greater than 2:1 molar ratio compared to rhodium.

### FIELD

The present invention relates to homogeneous olefin hydroformylation processes and in particular, to hydroformylation processes that utilize recycle streams with solubilized rhodium-phosphite complex catalysts.

### BACKGROUND

It is known in the art that aldehydes may be readily produced by reacting an olefinically unsaturated compound with carbon monoxide and hydrogen in the presence of a solubilized rhodium-phosphite ligand complex catalyst and that a preferred type of such processes involves continuous hydroformylation and recycling of the catalyst, such as disclosed, e.g., in U.S. Pat. No. 4,599,206. Often, such processes utilize liquid recycle, although batch processes or gas recycle hydroformylation processes are also feasible.

Despite the benefits attendant with such solubilized rhodium-phosphite complex catalyzed liquid recycle hydroformylation processes, under certain circumstances, the rhodium in some rhodium-phosphite complex catalysts may lose activity and/or precipitate from solution during hydroformylation as rhodium metal or in the form of clusters of rhodium. Rhodium is very expensive thus loss of activity or rhodium from solution is a severe economic issue for commercial use of these catalysts.

The use of stripping gas vaporizers including those with added CO to mitigate rhodium losses during the vaporization stages have been reported (see, e.g., U.S. Pat. No. 8,404,903 and PCT Publication WO2016/089602). However, such systems require significant capital outlay and are not readily retrofitted to an existing facility.

The use of polymeric additives containing polar functional groups has been found to mitigate the rhodium loss as reported in U.S. Pat. Nos. 4,774,361 and 11,111,198. However, these additives merely preserve activity.

A number of reports of using mixed transition metal homogeneous catalyst systems claim improved catalyst performance. For example, U.S. Pat. No. 4,262,141 teaches hydroformylation with Rh/phosphine catalysts in the presence of copper, silver, or zinc additives. U.S. Pat. No. 5,756,855A employs a Group VIII metal (other than rhodium) for stabilization of a rhodium/monophosphite catalyst and U.S. Pat. No. 4,306,086A teaches stabilizing and regenerating hydroformylation catalysts based on rhodium/triarylphosphine complexes by adding cobalt compounds. U.S. Pat. No. 4,200,592A teaches that the addition of homogeneous Group VI or Group VIII metal co-catalyst increases the activity of homogeneous rhodium-based hydroformylation of internal olefin(s) at low pressures using a rhodium/organophosphorous catalyst. They reported that Group VII metals had no effect on the rhodium/triarylphosphine-based catalyst performance.

It would be desirable to have alternative approaches for improving rhodium/phosphite-based homogenous hydroformylation catalyst activity. This would enable lower rhodium costs and/or lower reaction temperatures which may promote longer catalyst life and lower heavies formation.

### SUMMARY

The present invention has advantageously discovered an alternative approach for increasing catalyst activity in hydroformylation processes that utilize a recycle stream of rhodium-phosphite complex catalysts. Some embodiments of the present invention advantageously provide hydroformylation processes with an improved solubilized rhodium-phosphite complex catalyzed liquid recycle operation of olefins (C₃ and higher) wherein the activity of the rhodium-phosphite catalyst is increased, thus reducing the amount of rhodium catalyst needed to generate a specific aldehyde production rate. Alternatively, the higher activity may allow lower reaction temperatures, which may promote longer catalyst life, lower ligand degradation, lower aldehyde condensation reactions (i.e., heavies formation), or other undesirable side reactions.

In one aspect, a homogeneous olefin hydroformylation process for producing an aldehyde comprises (1) contacting in a reaction zone reactants comprising an (a) olefin, (b) hydrogen and (c) CO, in the presence of (d) a catalytic amount of soluble rhodium-monophosphite based catalyst, optionally with free organophosphite ligand, wherein the monophosphite is one in which each phosphorus atom is bonded to three oxygen atoms and at least one such oxygen atom is bonded to a carbon atom of an aromatic ring that is adjacent to another carbon atom of said ring having a pendant monovalent group (hindering group), having a steric hindrance at least as great as the steric hindrance of the isopropyl group, and (e) a Group VII metal complex additive in an amount sufficient to increase the rate of the hydroformylation process; and (2) maintaining the reaction mixture under conditions at which the olefinic compound reacts with the hydrogen and carbon monoxide to form an aldehyde.

In marked contrast to the findings in U.S. Pat. No. 4,200,592, the use of Group VII catalyst precursors had a profound effect on reaction rate with monophosphite-based catalysts (as opposed to monophosphine-based catalysts). Without being bound by theory, this surprising difference is presumably related to the stronger binding characteristics of phosphines compared to phosphites.

These and other embodiments are discussed in more detail in the Detailed Description below.

### DETAILED DESCRIPTION

All references to the Periodic Table of the Elements and the various groups therein are to the version published in the CRC Handbook of Chemistry and Physics, 72nd Ed. (1991-1992) CRC Press, at page I-11.

Unless stated to the contrary, or implicit from the context, all parts and percentages are based on weight and all test methods are current as of the filing date of this application. For purposes of U.S. patent practice, the contents of any referenced patent, patent application or publication are incorporated by reference in their entirety (or its equivalent US version is so incorporated by reference) especially with respect to the disclosure of definitions (to the extent not inconsistent with any definitions specifically provided in this disclosure) and general knowledge in the art.

As used herein, “a,” “an,” “the,” “at least one,” and “one or more” are used interchangeably. The terms “comprises,” “includes,” and variations thereof do not have a limiting meaning where these terms appear in the description and claims. Thus, for example, an aqueous composition that includes particles of “a” hydrophobic polymer can be interpreted to mean that the composition includes particles of “one or more” hydrophobic polymers.

Also herein, the recitations of numerical ranges by endpoints include all numbers subsumed in that range (e.g., 1 to 5 includes 1, 1.5, 2, 2.75, 3, 3.80, 4, 5, etc.). For the purposes of the invention, it is to be understood, consistent with what one of ordinary skill in the art would understand, that a numerical range is intended to include and support all possible subranges that are included in that range. For example, the range from 1 to 100 is intended to convey from 1.01 to 100, from 1 to 99.99, from 1.01 to 99.99, from 40 to 60, from 1 to 55, etc. Also herein, the recitations of numerical ranges and/or numerical values, including such recitations in the claims, can be read to include the term “about.” In such instances the term “about” refers to numerical ranges and/or numerical values that are substantially the same as those recited herein.

As used herein, the terms “ppm” and “ppmw” are used interchangeably and mean parts per million by weight.

For purposes of this invention, the term “hydrocarbon” is contemplated to include all permissible compounds having at least one hydrogen and one carbon atom. Such permissible compounds may also have one or more heteroatoms. In a broad aspect, the permissible hydrocarbons include acyclic (with or without heteroatoms) and cyclic, branched and unbranched, carbocyclic and heterocyclic, aromatic and nonaromatic organic compounds that can be substituted or unsubstituted.

As used herein, the term “substituted” is contemplated to include all permissible substituents of organic compounds unless otherwise indicated. In a broad aspect, the permissible substituents include acyclic and cyclic, branched and unbranched, carbocyclic and heterocyclic, aromatic and nonaromatic substituents of organic compounds. Illustrative substituents include, for example, alkyl, alkyloxy, aryl, aryloxy, hydroxyalkyl, aminoalkyl, in which the number of carbons can range from 1 to 20 or more, preferably from 1 to 12, as well as hydroxy, halo, and amino. The permissible substituents can be one or more and the same or different for appropriate organic compounds. This invention is not intended to be limited in any manner by the permissible substituents of organic compounds.

As used herein, the terms “hydroformylation” or “hydroformylation process” are contemplated to include, but are not limited to, all hydroformylation processes that involve converting one or more substituted or unsubstituted olefinic compounds or a reaction mixture comprising one or more substituted or unsubstituted olefinic compounds to one or more substituted or unsubstituted aldehydes or a reaction mixture comprising one or more substituted or unsubstituted aldehydes. The aldehydes may be asymmetric or non-asymmetric.

The terms “reaction fluid,” “reaction medium” and “catalyst solution” are used interchangeably herein, and may include, but are not limited to, a mixture comprising: (a) a metal-organophosphorous ligand complex catalyst, (b) free organophosphorous ligand, (c) aldehyde product formed in the reaction, (d) unreacted reactants, (e) a solvent for said metal-organophosphorous ligand complex catalyst and said free organophosphorous ligand, and, optionally, (f) one or more phosphorus acidic compounds, which may be dissolved and/or suspended, formed in the reaction. The reaction fluid can encompass, but is not limited to, (a) a fluid in a reaction zone, (b) a fluid stream on its way to a separation zone, (c) a fluid in a separation zone, (d) a recycle stream, (e) a fluid withdrawn from a reaction zone or separation zone, (f) a withdrawn fluid being treated with an acid removal system such as an extractor or other immiscible fluid contacting system, (g) a treated or untreated fluid returned to a reaction zone or separation zone, (h) a fluid in an external cooler, and (i) ligand decomposition products and components derived from them, such as oxides, sulfides, salts, oligomers, and the like.

“Organomonophosphite ligands” are compounds containing a single phosphorous atom bound to three oxygen atoms; the three oxygen atoms are each additionally bound to carbon moieties. At least one such oxygen atom is bonded to a carbon atom of an aromatic ring that is adjacent to another carbon atom of said ring having a pendant monovalent group (hindering group) having a steric hindrance at least as great as the steric hindrance of the isopropyl group. Illustrative examples include, but are not limited to monoorganophosphite, diorganophosphite, triorganophosphite compounds, examples of which include: tris(2,4-di-t-butylphenyl)phosphite, 4,8-di-tert-butyl-6-(2-(tert-butyl)-4-methoxyphenyl)-2,10 dimethoxydibenzo[d,f][1,3,2]dioxaphosphepine, and the like.

The term “free ligand” means ligand that is not complexed with (or bound to) the metal, e.g., metal atom, of the complex catalyst.

For the purposes of this invention, the terms “heavy byproducts” and “heavies” are used interchangeably and refer to liquid byproducts that have a normal boiling point that is at least 25° C. above the normal boiling point of the desired product of the hydroformylation process. Such materials are known to form in hydroformylation processes under normal operation through one or more side reactions, including for example, by aldol condensation.

For the purpose of this invention, the term “dimer” when referring to heavy byproducts from a hydroformylation reaction refers to heavy byproducts derived from two molecules of aldehyde. Likewise, the term “trimer” when referring to heavy byproducts from a hydroformylation reaction refers to heavy byproducts derived from three molecules of aldehyde.

For the purposes of this invention, the terms “separation zone” and “vaporizer” are used interchangeably and refer to a separation device wherein the product aldehyde is typically volatilized overhead, condensed and collected, while the non-volatile concentrated effluent (tails, or vaporizer tails) containing the homogeneous catalyst is returned to one or more of the reactors. The vaporizer temperature is typically higher than the reactor temperature and may optionally be operated at reduced pressure. In one embodiment, the vaporizer features flowing gas of varying composition that aids in product removal and optionally helps stabilize the catalyst (“strip gas vaporizer”). Other separation zone processes such as liquid/liquid extraction or membrane filtration may also be employed.

The homogeneous olefin hydroformylation process for producing an aldehyde of the present invention comprises:
(1) contacting in a reaction zone, reactants comprising an (a) olefin, (b) hydrogen and (c) CO, in the presence of (d) a catalytic amount of soluble rhodium-monophosphite based catalyst, optionally with free organophosphite ligand, wherein the monophosphite is one in which each phosphorus atom is bonded to three oxygen atoms and at least one such oxygen atom is bonded to a carbon atom of an aromatic ring that is adjacent to another carbon atom of said ring having a pendant monovalent group (hindering group), having a steric hindrance at least as great as the steric hindrance of the isopropyl group, and (e) a Group VII metal complex additive in an amount sufficient to increase the rate of the hydroformylation process; and
(2) maintaining the reaction mixture under conditions at which the olefinic compound reacts with the hydrogen and carbon monoxide to form an aldehyde.

In preferred embodiments, the Group VII metal additive complex comprises a metal complex in a low oxidation state, preferably no more than +2, and most preferably no more than +1. Although higher oxidation state additives may be reduced under the hydroformylation conditions, the presence of such high oxidation state materials may cause heavies formation and/or aldehyde oxidation thus are not preferred.

The preferred ligands on the Group VII metal additive complex are carbon and phosphorous based such as CO, acetylacetonate, and organophosphorous ligands (most preferably organophosphite ligands). The ligands are preferably substantially free of cyanide, sulfur, or halogens. The Group VII metal additives are preferably soluble in the organic matrix of the catalyst solution thus non-ionic (neutral) complexes are generally preferred. In general, the most preferred and readily available Group VII metal additives are the Mx (CO) y family of compounds.

The preferred group VII metal (M) is manganese. A preferred Group VII metal complex additive for some embodiments is Mn₂(CO)₁₂.

Under the hydroformylation conditions, the Group VII metal additive may undergo derivatization reactions such as M-M bond cleavage or substitution of one or more CO groups with monophosphites. The exact structure of the Group VII additive under hydroformylation conditions is not known.

The amount of Group VII metal complex additive is not narrowly critical but preferably is above 2:1 molar ratio compared to the rhodium metal. Above a ratio of 20:1 is not generally advantageous and may promote higher heavies formation. In some embodiments it may be advantageous to add the Group VII metal complex additive in a ratio of from 5:1 to 15:1, or even 8:1 to 12:1, compared to the rhodium metal.

In general such hydroformylation reactions involve the production of aldehydes by reacting an olefinic unsaturated compound with carbon monoxide and hydrogen in the presence of a solubilized rhodium-phosphite complex catalyst in a liquid medium that also contains a solvent for the catalyst, and free phosphite ligand (i.e. ligand that is not complexed with the rhodium metal in the active complex catalyst). The recycle procedure generally involves withdrawing a portion of the liquid reaction medium containing the catalyst and aldehyde product from the hydroformylation reaction zone, either continuously or intermittently, and distilling the aldehyde product therefrom in one or more stages under normal, reduced or elevated pressure, as appropriate, in a separate distillation zone in order to recover the aldehyde product and other volatile materials in vaporous form, the non-volatilized rhodium catalyst containing residue being recycled to the reaction zone. Condensation of the volatilized materials, and separation and recovery thereof, e.g. by distillation, can be carried out in any conventional manner, the aldehyde product being passed on for further purification if desired and any recovered reactants e.g. olefinic starting material and syngas recycled in any desired manner to the hydroformylation zone. Likewise, the recovered non-volatilized rhodium catalyst containing residue can be recycled with or without further treatment to the hydroformylation zone in any conventional manner desired. Accordingly, the general hydroformylation processes in which embodiments of the present invention may be implemented correspond to any of the known processing techniques heretofore employed in conventional gas or liquid catalyst recycle hydroformylation reactions.

Illustrative rhodium-phosphite complex catalysts employable in such hydroformylation reactions encompassed by this invention may include, without limitation, those disclosed in the above mentioned patents and applications. In general such catalysts may be pre-formed or formed in situ as described in such references and consist essentially of rhodium in complex combination with an organophosphite ligand. It is believed that carbon monoxide is also present and complexed with the rhodium in the active species. The active catalyst species may also contain hydrogen directly bonded to the rhodium.

Illustrative organophosphite ligands that may be employed as the organophosphite ligand complexed to the rhodium catalyst and/or free organophosphite ligand in such hydroformylation reactions encompassed by this invention may include a variety of tertiary organophosphites, such as preferably diorganophosphites of the formula (III) wherein, R¹ represents a divalent organic radical and W represents a substituted or unsubstituted monovalent hydrocarbon radical.

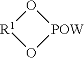

Representative divalent radicals represented by R¹ in Formula (III) above include those wherein R¹ may be a divalent acyclic radical or a divalent aromatic radical. Illustrative divalent acyclic radicals are e.g. alkylene, alkylene-oxy-alkylene, alkylene-N_{X}-alkylene wherein is hydrogen or a monovalent hydrocarbon radical, alkylene-S-alkylene, and cycloalkylene radicals; and the like, such as disclosed more fully e.g. in U.S. Pat. Nos. 3,415,906 and 4,567,306, and the like. Illustrative divalent aromatic radicals are e.g. arylene, bi-arylene, arylene-alkylene, arylene alkylene-arylene, arylene-oxy-arylene, arylene-oxy-alkylene, arylene-NX-arylene and arylene NX-alkylene wherein X is hydrogen or a monovalent hydrocarbon radical, arylene-S-alkylene, and arylene-S-arylene radicals; and the like. More preferably R¹ is a divalent aromatic radical.

Representative of a more preferred class of tertiary diorganophosphites are diorganophosphites of the formula (IV) wherein W is a substituted or unsubstituted monovalent hydrocarbon radical, Ar is a substituted or unsubstituted aryl radical, each Ar being the same or different, each y individually has a value of 0 or 1, Q is a divalent bridging group selected from the group consisting of —CR³R⁴—, —O—, —S—, —NR⁵—, SiR⁶R⁷— and —CO—, wherein each R³ and R⁴ is independently selected from the group consisting of hydrogen, alkyl radicals having 1 to 12 carbon atoms, phenyl, tolyl and anisyl, wherein each R⁵, R⁶ and R⁷ are independently hydrogen or a methyl radical, and n has a value of 0 or 1. Formula (IV) type diorganophosphites are described in greater detail, e.g., in U.S. Pat. Nos. 4,599,206 and 4,717,775.

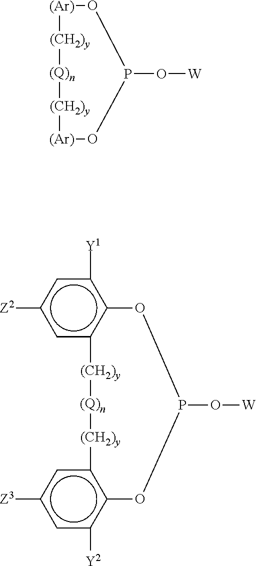

Among the more preferred diorganophosphites are those of the formula (V) wherein Q is —CR¹R² and each R¹ and R² radical individually represents a radical selected from the group consisting of hydrogen and alkyl; wherein each y individually has a value of 0 or 1, and n has a value of 0 to 1; wherein W represents in unsubstituted or substituted monovalent hydrocarbon radical selected from the group consisting of alkyl radicals having from 1 to 36 carbon atoms, (such as primary, secondary and tertiary alkyl radicals e.g. methyl, ethyl, n-propyl, isopropyl, butyl, sec-butyl, t-butyl, t-butylethyl, t butylpropyl, n-hexyl, amyl, sec-amyl, t-amyl, iso-octyl, 2-ethylhexyl, decyl, octadecyl, and the like) as well as, aryl radicals, such as alpha-naphthyl, beta-naphthyl, and aryl radicals of the formula (VI):

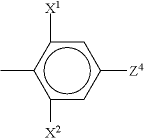

and wherein each X¹, X², Y¹, Y², Z², Z³, and Z⁴ group individually represents a radical selected from the group consisting of hydrogen, an alkyl radical having from 1 to 8 carbon atoms, substituted or unsubstituted aryl, alkaryl, aralkyl and alicyclic radicals (e.g. phenyl, benzyl, cyclohexyl, 1-methylcyclohexyl, and the like), hydroxy (—OH), and an ether (i.e oxy) radical such as —OR⁸ wherein R⁸ is an alkyl radical of 1 to 18 carbon atoms. Among other diorganophosphites are those of Formula (V) above as described in U.S. Pat. Nos. 4,599,206, 4,717,775, and WO 2016/087301.

Another group of tertiary organophosphites that may be employed in such hydroformylation reactions encompassed by this invention are tertiary mono organophosphites of the formula (VII) wherein Z⁵ represents a trivalent organic radical, such as described in greater detail e.g. in U.S. Pat. No. 4,567,306.

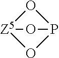

Finally another group of tertiary organophosphites that may be employed in such hydroformylation reactions encompassed by this invention include triorganophosphites, such as tris(2,4-di-t-butylphenyl)phosphite, tris(ortho-phenyl)phenyl phosphite, tris(ortho-methyl)phenyl phosphite, tris(ortho-t-butyl)phenyl phosphite, and the like. Such triorganophosphites are described in greater detail, for example, in U.S. Pat. Nos. 3,527,809, 4,717,775 and 9,737,884.

Thus the organophosphite ligand employable in the hydroformylation reactions encompassed by this invention as the organophosphite ligand of the rhodium-organophosphite complex catalyst and/or as the free organophosphite ligand present in the hydroformylation reaction medium and liquid solutions throughout the hydroformylation process may be a tertiary organic phosphite ligand selected from the group consisting of monoorganophosphites, diorganophosphites, and triorganophosphites, such as described above. Mixtures of tertiary organic phosphite ligands may be employed as well.

The hydroformylation process encompassed by this invention may be carried out in any excess amount of free organophosphite ligand desired, e.g. at least one mole of free organophosphite ligand per mole rhodium present in the reaction medium on up to 100 moles of free organophosphite ligand or higher if desired. In general amounts of organophosphite ligand of from about 4 to about 50 moles per mole rhodium present in the reaction medium should be suitable for most purposes, said amounts being the sum of both the amount of organophosphite that is bound (complexed) to the rhodium present and the amount of free (non-complexed) organophosphite ligand present. Of course, if desired, make-up organophosphite ligand can be supplied to the reaction medium of the hydroformylation process, at any time and in any suitable manner, to maintain a predetermined level of free ligand in the reaction medium. Moreover, it is to be understood that while the organophosphite ligand of the rhodium-organophosphite complex catalyst and excess free organophosphite ligand in a given process are both normally the same, different organophosphite ligands, as well as, mixtures of two or more different organophosphite ligands may be employed for each purpose in any given process, if desired.

The amount of rhodium-organophosphite complex catalyst present in the reaction medium of a given hydroformylation process encompassed by this invention need only be that minimum amount necessary to provide the given rhodium concentration desired to be employed and which will furnish the basis for at least that catalytic amount of rhodium necessary to catalyze the particular hydroformylation process involved such as disclosed e.g. in the above-mentioned patents and applications. In general, rhodium concentrations in the range of from about 10 ppm to about 1000 ppm, calculated as free rhodium, in the hydroformylation reaction medium should be sufficient for most processes, while it is generally preferred to employ from about 5 to 500 ppm of rhodium and more preferably from 10 to 350 ppm to rhodium.

The olefinic starting material reactants that may be employed in the hydroformylation reactions encompassed by of this invention can be terminally or internally unsaturated and be of straight-chain, branched-chain or cyclic structure, such as disclosed e.g. in the above-mentioned patents and applications. Such olefins can contain from 2 to 20 carbon atoms and may contain one or more ethylenic unsaturated groups. Moreover, such olefins may contain groups or substituents which do not essentially adversely interfere with the hydroformylation process such as carbonyl, carbonyloxy, oxy, hydroxy, oxycarbonyl, halogen, alkoxy, aryl, alkyl, haloalkyl, and the like. Illustrative olefinic unsaturated compounds include alpha olefins, internal olefins, alkyl alkenoates, alkenyl alkanoates, alkenyl alkyl ethers, alkenols, and the like, e.g. ethylene, propylene, 1-butene, 1-pentene, 1-hexene, 1-octene, 1-decene, 1-dodecene, 1-octadecene, 2-butene, isobutylene, 2-methylbutene, 2-hexene, 3-hexene, 2-heptene, cyclohexene, propylene dimers, propylene trimers, propylene tetramers, butene dimers, butene trimers, 2-ethyl-1-hexene, styrene, 3-phenyl-1-propene, 1,4-hexadiene, 1,7-octadiene, 3-cyclohexyl-1-butene, and the like. Of course, it is understood that mixtures of different olefinic starting materials can be employed, if desired. Embodiments of the present invention can be particularly useful in the hydroformylation of C₄ and higher olefins. Thus, in some embodiments, the olefinic unsaturated starting materials are alpha olefins containing from 3 to 20 carbon atoms, and internal olefins containing from 4 to 20 carbon atoms as well as starting material mixtures of such alpha olefins and internal olefins.

As noted above hydroformylation reactions encompassed by this invention are also conducted in the presence of an organic solvent for the rhodium-phosphite complex catalyst. Any suitable solvent which does not unduly adversely interfere with the intended hydroformylation process can be employed. Illustrative suitable solvents for rhodium catalyzed hydroformylation processes include those disclosed e.g. in the above mentioned patents and applications. Of course mixtures of one or more different solvents may be employed if desired. Most preferably the solvent will be one in which the olefinic starting material, hydroformylation catalyst and organic polymer additive employed herein are all substantially soluble. In general, it is preferred to employ aldehyde compounds corresponding to the aldehyde products desired to be produced and/or higher boiling aldehyde liquid condensation by products as the primary solvent such as the higher boiling aldehyde liquid condensation by-products that are produced in situ during the hydroformylation process. Indeed, while one may employ any suitable solvent at the startup of a continuous process, the primary solvent will normally eventually comprise both aldehyde products and higher boiling aldehyde liquid condensation by products due to the nature of such continuous processes. Such aldehyde condensation by-products can also be pre-formed if desired and used accordingly. These condensation products contain polar moieties such as esters and alcohols yet do not appear to stabilize the rhodium catalysts from generating clusters and colloids. Of course, the amount of solvent employed is not critical to the subject invention and need only be that amount sufficient to provide the reaction medium with the particular rhodium concentration desired for a given process. In general, the amount of solvent when employed may range from about 5 percent by weight up to about 95 percent by weight or more based on the total weight of the reaction medium.

The hydroformylation reaction conditions that may be employed in the hydroformylation processes encompassed by this invention may include any suitable continuous liquid catalyst recycle hydroformylation conditions heretofore disclosed in the above-mentioned patents and applications. For instance, the total gas pressure of hydrogen, carbon monoxide and olefinic unsaturated starting compound of the hydroformylation process may range from about 7 to about 69000 kPa(a). In general, however, it is preferred that the process be operated at a total gas pressure of hydrogen, carbon monoxide and olefinic unsaturated starting compound of less than about 10300 kPa(a) and more preferably less than about 3400 kPa(a). The minimum total pressure being limited predominately by the amount of reactants necessary to obtain a desired rate of reaction. More specifically the carbon monoxide partial pressure of the hydroformylation process of this invention is preferably from about 7 to about 830 kPa(a). and more preferably from about 21 to about 620 kPa(a), while the hydrogen partial pressure is preferably about 100 to about 1100 kPa(a) and more preferably from about 200 to about 690 kPa(a). In general H₂:CO molar ratio of gaseous hydrogen to carbon monoxide may range from about 1:10 to 100:1 or higher, the more preferred hydrogen to carbon monoxide molar ratio being from about 1:1 to about 10:1. Further, the hydroformylation process may be conducted at a reaction temperature from about 45° C. to about 150° C. In general, hydroformylations at reaction temperatures of about 50° C. to about 120° C. are preferred for all types of olefinic starting materials; higher temperatures are considered to be less desirable, due to possible catalyst activity decline as disclosed e.g., in U.S. Pat. No. 4,599,206.

Moreover, as noted herein, the solubilized rhodium-phosphite complex catalyzed continuous hydroformylation process employable in this invention involves a liquid catalyst recycle procedure. Such types of liquid catalyst recycle procedures are known as disclosed e.g. in the above-mentioned patents and applications, and thus need not be particularly detailed herein, since any such conventional catalyst recycle procedures may be employed by this invention. For instance in such liquid catalyst recycle procedures it is common place to continuously remove a portion of the liquid reaction product medium, containing e.g. the aldehyde product, the solubilized rhodium-phosphite complex catalyst, free phosphite ligand, and organic solvent, as well as by-products produced in situ by the hydroformylation, e.g. aldchyde condensation by-products etc., and unreacted olefinic starting material, carbon monoxide and hydrogen (syn gas) dissolved in said medium, from the hydroformylation reactor, to a distillation zone, e.g. a vaporizer/separator wherein the desired aldehyde product is distilled in one or more stages under normal, reduced or elevated pressure as appropriate and separated from the liquid medium. The vaporized or distilled desired aldehyde product so separated may then be condensed and recovered in any conventional manner as discussed above. The remaining non-volatilized liquid residue which contains rhodium-phosphite complex catalyst, solvent, free phosphite ligand and usually some aldehyde product is then recycled back, with or without further treatment as desired, along with whatever by-product and non-volatilized gaseous reactants that might still also be dissolved in said recycled liquid residue, in any conventional manner desired, to the hydroformylation reactor, such as disclosed e.g. in the above-mentioned patents and applications. Moreover, the reactant gases so removed by such distillation from the vaporizer may also be recycled back to the reactor if desired.

The distillation and separation of the desired aldehyde product from the rhodium-phosphite complex catalyst containing product solution may take place at any suitable temperature desired. In general, it is recommended that such distillation take place at low temperatures, such as below 150° C., preferably below 140° C., and more preferably at a temperature in the range of from about 50° C. to about 130° C. Such aldehyde distillation generally takes place under reduced pressure, e.g. a total gas pressure that is substantially lower than the total gas pressure employed during hydroformylation when low boiling aldehydes (e.g. C₄ to C₆) are involved or under vacuum when high boiling aldehydes (e.g. C₇ or greater) are involved. For instance, a common practice is to subject the liquid reaction product medium removed from the hydroformylation reactor to a pressure reduction so as to volatilize a substantial portion of the unreacted gases dissolved in the liquid medium and then pass said volatilized gases and liquid medium which now contains a much lower syn gas concentration than was present in the hydroformylation reaction medium to the distillation zone e.g. vaporizer/separator, wherein the desired aldehyde product is distilled. In general distillation pressures ranging from vacuum pressures or below on up to total gas pressures of about 50 psig should be sufficient for most purposes.

In some embodiments, the polymer additives described in U.S. Pat. Nos. 4,774,361 and 11,111,198 may be used. The amount of such organic polymer additives employable in any given process of this invention need only be that minimum amount necessary to furnish the basis for at least some minimization of such rhodium loss that might be found to occur as a result of carrying out an identical rhodium catalyzed liquid recycle hydroformylation process under identical conditions, save for carrying out said identical process in the absence of the identical organic polymer employed in said given process. Amounts of such organic polymer additives ranging from about 0.1 up to about 3 weight percent based on the total weight of the fluid in the hydroformylation reaction zone should be sufficient for most processes. The upper amount of organic polymer additive employable herein is governed primarily by the solubility limit of the organic polymer in the non-volatilized liquid rhodium catalyst containing residue obtained after distillation removal of as much of the aldehyde product desired. In general, the amount of such organic polymer additives is in the range of about 0.1 to about 3.0 with about 0.25 to about 2.5 weight percent being desirable in some embodiments, each based on the total weight of the fluid in the hydroformylation reaction zone.

The addition of the Group VII metal complex additives employable in this invention to the hydroformylation reaction fluid may be carried out in any suitable manner desired. For instance, the Group VII complex may be added to the reaction fluid at any time prior to or during the initiation of the aldehyde production. In general, it is preferred to add such Group VII metal complex additives directly to the hydroformylation reaction fluid and allow the Group VII metal complex additive to remain in solution throughout the entire liquid catalyst recycle hydroformylation solution.

Some embodiments of the invention will now be described in more detail in the following Examples.

### EXAMPLES

All parts and percentages in the following examples are by weight unless otherwise indicated. Pressures are given as absolute pressure unless otherwise indicated.

### General Procedure: Single Pass Hydroformylation Procedure:

Hydroformylation experiments were performed (glass reactor) in order to compare the reaction rate of tris-(2,4-ditertbutylphenyl)phosphite (Ligand A) in the presence or absence of group VII metal. To evaluate catalyst performance, 90 ml Fisher Porter bottles that are equipped with a sampling port, inlet/outlet valve, and pressure gauge are used as reaction vessels. The Fisher Porter bottles are initially inerted with N₂ unless otherwise indicated and are heated in a temperature controlled oil bath. A solution of 25 ppm rhodium and 2 wt % Ligand A in tetraglyme was charged and operated under the conditions described below in Table 1.

After sealing the reactor, the system is purged with nitrogen and the oil bath is heated to furnish the desired hydroformylation reaction temperature. The hydroformylation reaction is conducted at a total pressure of 150 to 160 psig (1034 to 1103 kPa) and at a temperature ranging from 60 to 100° C. A feed comprising nitrogen, syngas, and propylene is started. The flows of the feed gases (H₂, CO, propylene, N₂) are controlled individually with mass flow meters as indicated in Table 1 and the feed gases are dispersed in the catalyst precursor solution via fritted metal spargers. The partial pressures of N₂, H₂, CO, propylene, and aldehyde products are determined by analyzing the vent stream by GC analysis and Dalton's Law. The unreacted portion of the feed gases is stripped out with butyraldehydes products by the nitrogen flow to maintain substantially constant liquid level. Flows and feed gas partial pressures are set to obtain hydroformylation reaction rates of around 2 gram-moles aldehyde per liter reaction fluid per hour. In practice, it is often observed that the system takes about one day to arrive at steady state conditions due to removing trace air from feed lines and reaching thermal equilibration of oil baths or other substantial changes in process variables.

The reaction was started in the absence of group VII metal. After 5 days, dimanganese dodecacarbonyl was added at Mn/Rh mol ratio of 10:1 as a solution in Toluene. The results are presented below:

TABLE 1
Operating conditions for Single Pass
Hydroformylation Performance testing.

| | | Ligand A/Rh | Target rate | |
|---|---|---|---|---|
| Solvent | Ligand | molar Ratio | (g-mol/l-hr) | Olefin |
| tetraglyme | Ligand A | 120 | 1.5-2 | propylene |

| Rh | Temp | CO | H2 | Olefin | Total flow |
|---|---|---|---|---|---|
| (ppm) | (° C.) | (psi) | (psi) | (psi) | (sL/hr) |
| 25 | 75 | 50 | 50 | 5 | 30 |

The initial period is typical of this fresh catalyst system in the absence of known poisons or inhibitors. The rate increases as soon as manganese compound is added and remains stable which resulted in a decrease in propylene partial pressure. Since the rate is roughly first order in olefin concentration, to be able to compare rates, the olefin partial pressure was increased to that of the control (initial) period. After readjustment of the propylene partial pressure, the rate has increased from 1.6 gmol/L/Hr to 2.8 gmol/L/hr affording a 75% rate increase.

What is claimed is:

1. A homogeneous olefin hydroformylation process for producing an aldehyde, the process comprising: contacting in a reaction zone, reactants comprising an olefin, hydrogen and CO in the presence of a rhodium-organophosphite based catalyst, in the presence of a soluble Group VII metal complex additive, wherein the Group VII complex additive is present at greater than 2:1 molar ratio compared to rhodium.

2. The process of claim 1 wherein the olefin has four or more carbon atoms.

3. The process of claim 1 wherein the olefin has eight or more carbon atoms.

4. The process of claim 1 wherein the Group VII metal in the Group VII metal complex additive is manganese.

5. The process of claim 1 wherein the Group VII metal complex additive is non-ionic.

6. The process of claim 1 wherein the Group VII metal complex additive is substantially free of halogens, sulfur, and cyanide.

7. The process of claim 1 wherein the Group VII complex additive is added in an amount to have a ratio of Group VII complex additive to rhodium in a range of from 5:1 to 15:1.

8. The process of claim 1 where the reactants are contacted in the presence of free organophosphite ligand in addition to the rhodium-organophosphite based catalyst and the Group VII metal complex additive.
